(19) United States
(12) Patent Application Publication
(10) Pub. No.: US 2025/0135111 A1
(43) Pub. Date: May 1, 2025

(54) Method and Apparatus for Monitoring the Flow State in Infusion Tubing

(71) Applicant: Medcaptain Medical Technology Co., Ltd., Shenzhen (CN)

(72) Inventors: Xiang LI, Shenzhen (CN); Zhu XU, Shenzhen (CN); Haoke HUANG, Shenzhen (CN)

(73) Assignee: Medcaptain Medical Technology Co., Ltd., Shenzhen (CN)

(21) Appl. No.: 18/928,026

(22) Filed: Oct. 27, 2024

(30) Foreign Application Priority Data
Oct. 27, 2023 (CN) 202311419327.8

(51) Int. Cl. A61M 5/168; A61M 5/142

(52) U.S. Cl. CPC A61M 5/16831; A61M 5/142; A61M 2005/16868; A61M 2205/18; A61M 2205/3327; A61M 2205/3375; A61M 2205/50

(57) ABSTRACT

The flow state of infusion tubing is monitored by measuring the received strength of ultrasound transmitted through the tubing at at least two moments during operation of an infusion device. The flow state may be a normal state, a blocked state, or an air bubble state. Which state is present is determined by not only a degree of signal attenuation within the tubing, but also by the rate of change of attenuation.

### REFERENCE TO RELATED APPLICATIONS

The invention claims priority of Chinese Patent Application No. 202311419327.8, entitled “Method, Device, and Apparatus for Detecting Infusion Tubing, and Computer-Readable Storage Medium” filed with the China National Intellectual Property Administration on Oct. 27, 2023, which is incorporated herein by reference in its entirety.

### TECHNICAL FIELD

This invention generally relates to medical devices, and more particularly, to a method, device, and apparatus for monitoring the flow state within infusion tubing.

### BACKGROUND ART

Infusion devices can be used for delivering fluids such as medication and nutrient solutions. During the process of fluid delivery, the infusion tubing may become blocked or air bubbles may form. In order to ensure stable operation of the infusion device, it is necessary to monitor the condition of the fluid inside the infusion tubing.

Currently, the detection of blockages and bubbles in the infusion tubing is performed using a blockage sensor and an air bubble sensor, which detect these issues separately. The need for multiple sensors complicates the detection process and increases the production cost of the infusion pump.

### SUMMARY OF THE INVENTION

The invention provides a method and a device for monitoring the flow state within infusion tubing, which solve the problem of complex structure and high cost in the infusion pumps that incorporate blockage and air bubble detection in the related art.

In a first aspect, the invention provides a method for monitoring the flow state of infusion tubing used in an infusion system, which comprises an infusion device and a data acquisition device communicatively connected to the infusion device, the data acquisition device being disposed on infusion tubing of the infusion device; wherein the method comprises:
transmitting ultrasound from a transmitting portion of the data acquisition device through the infusion tubing;
receiving the ultrasound in a receiving portion of the data acquisition device after the ultrasound has passed through the infusion tubing;
obtaining data indicting the signal strength of the received ultrasound acquired by the data acquisition device at at least two moments during operation of the infusion device; and
determining an infusion state of the infusion tubing according to the signal strength data acquired by the data acquisition device at the at least two moments, wherein the infusion state comprises a normal state, a blocked state or an air bubble state.

In some embodiments, the data acquisition device is an ultrasound transducer.

In some embodiments, the step of determining the infusion state of the infusion tubing according to the signal strength data acquired by the data acquisition device at the at least two moments comprises:
when the signal strength data acquired by the data acquisition device at a first moment in time is less than or equal to a first data threshold, that is, a first received signal strength threshold, determining the infusion state of the infusion tubing according to a change in the signal strength data acquired by the data acquisition device after the first moment.

In some embodiments, the step of determining the infusion state of the infusion tubing according to the change in the signal strength data acquired by the data acquisition device after the first moment comprises:
determining an infusion state of the infusion tubing according to a rate of change of signal strength data acquired by the data acquisition device after the first moment.

In some embodiments, the step of determining the infusion state of the infusion tubing according to the change in the signal strength data acquired by the data acquisition device after the first moment comprises:
obtaining at least one of a number and a duration of signal strength data measurements acquired by the data acquisition device after the first moment that located between a first data threshold and a second data (corresponding to a received signal strength) threshold; and
determining the infusion state of the infusion tubing according to at least one of the number and the duration of signal strength data measurements located between the first data threshold and the second data threshold.

In some embodiments, the step of determining the infusion state of the infusion tubing according to the change in the signal strength data acquired by the data acquisition device after the first moment comprises:
determining the infusion state of the infusion tubing according to a difference between the signal strength data acquired by the data acquisition device after the first moment and the first data threshold.

In some embodiments, the method of monitoring the flow state of infusion tubing further comprises:
outputting an alarm message or a prompt message corresponding to the infusion state.

In a second aspect, the invention provides an apparatus for monitoring the flow state of infusion tubing, comprising:
an acquisition module configured to obtain signal strength data acquired by a data acquisition device during operation of the infusion device; and
a data analysis module configured to determine an infusion state of the infusion tubing according to the signal strength data acquired by the data acquisition device at at least two moments, wherein the infusion state comprises a normal state, a blocked state, or an air bubble state.

In a third aspect, the invention provides a nutrient infusion device comprising: at least one processor and a memory;

the memory is configured to store computer-executable instructions; and

the at least one processor is configured to process the computer-executable instructions stored by the memory to cause the nutrient infusion device to implement the method for monitoring the flow state of infusion tubing described in the first aspect or either of the optional approaches of the first aspect.

In a fourth aspect, embodiments of the invention provide a computer-readable storage medium having computer instructions stored therein, wherein the computer instructions when processed by the processor are configured to process the computer-executable instructions to cause a nutrient infusion device to realize the method for monitoring the flow state of the infusion tubing described in the first aspect or either of the optional approaches of the first aspect.

In the method, apparatus, and device for monitoring the flow state of infusion tubing, and a computer-readable storage medium of the invention, the detection method is applied to an infusion system, which includes an infusion device and a data acquisition device. The data acquisition device is provided in infusion tubing of the infusion device. During the operation of the infusion device, the data acquisition device may acquire a state of a fluid flow in infusion tubing of the infusion device at at least two moments and form data. By processing the signal strength data acquired by the data acquisition device at the at least two moments, an infusion state of the infusion tubing may be determined, so that it can be determined whether the infusion tubing is in a normal state, a blocked state, or an air bubble state. The invention can realize the function of monitoring the flow state of the state of the infusion tubing by acquiring the data of the fluid flow state in the infusion tubing several times by a data acquisition device, which reduces the number of data acquisition devices and reduces the types of signal strength data acquired by the data acquisition devices, thus achieving the goal of simplifying the structure of the infusion system.

### BRIEF DESCRIPTION OF THE DRAWINGS

The description, purpose, feature and advantage of the embodiments of the present application will be made easier to understand by the following detailed description with reference to the accompanying drawings. In the accompanying drawings, a plurality of embodiments of the present application will be illustrated by way of example as well as in a non-limiting manner.

FIG. 1 shows a schematic diagram of an infusion system according to an embodiment of the invention.

FIG. 2 shows a schematic diagram of a data acquisition device and infusion tubing in the infusion system according to an embodiment of the invention.

FIG. 3 shows a flowchart of a method for monitoring the flow state of infusion tubing according to an embodiment of the invention.

FIG. 4 shows a schematic structural diagram of a device for monitoring the flow state of infusion tubing according to an embodiment of the invention.

FIG. 5 is a schematic diagram of the structure of a nutrient infusion device according to an embodiment of the invention.

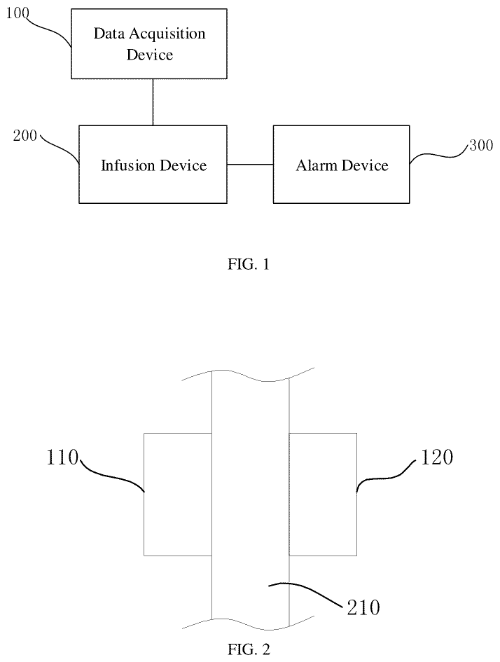

The accompanying drawings are labeled:
100—data acquisition device, 110—transmitting portion, 120—receiving portion;
200—infusion device, 210—infusion tubing;
300—alarm device;
400—detection device, 410—acquisition module, 420—data analysis module;
301—memory, 302—processor.

### DETAILED DESCRIPTION

Embodiments of the invention are described in detail below, and examples of the embodiments are shown in the accompanying drawings, where the same or similar labeling throughout denotes the same or similar elements or elements having the same or similar functions. The embodiments described below by reference to the accompanying drawings are examples only and are intended for use in explaining the invention and are not to be construed as limiting the application.

In the description of the invention, it is to be understood that the terms “center”, “longitudinal”, “lateral”, “length”, “width”, “thickness”, “top”, “bottom”, “front”, ‘back’, ‘left’, ‘right’, “vertical”, “horizontal”, “top”, “bottom”, “inside”, “outer”, ‘clockwise’, ‘counterclockwise’, ‘axial’, “radial”, ‘circumferential’, etc. indicate orientation or positional relationships according to those shown in the accompanying drawings, and are intended only to facilitate and simplify the description of the invention, and are not intended to indicate or imply that the device or element referred to must have a particular orientation, be constructed and operated in a particular orientation, and therefore cannot be construed as a limitation of the invention.

Furthermore, the terms “first” and “second” are used for descriptive purposes only and are not to be understood as indicating or implying relative importance or implicitly specifying the number of technical features indicated. Thus, a feature defined with the terms “first”, “second” may expressly or impliedly include at least one such feature. In the description of the invention, “plurality” means at least two, e.g., two, three, etc., unless otherwise expressly and specifically limited.

In this application, unless otherwise expressly specified and limited, the terms “mount”, “joint”, “connect”, “fix”, etc. are to be understood in a broad sense. For example, it may be a fixed connection, a removable connection, or an integral portion; it may be a mechanical connection, an electrical connection, or a communication with each other; it may be a direct connection or an indirect connection through an intermediate medium; it may be a connection within the two elements or an interaction between the two elements, unless expressly limited otherwise. For those of ordinary skill in the art, the specific meaning of the above terms in this application may be understood on a case-by-case basis.

In the invention, unless otherwise expressly provided and limited, the first feature “over” or “under” the second feature may be a direct contact between the first and second features, or an indirect contact between the first and second features through an intermediate medium. Furthermore, the first feature being “on” and “above” the second feature may be that the first feature is directly above or diagonally above the second feature, or simply that the first feature is horizontally higher than the second feature.

The descriptions “an embodiment”, “some embodiments”, “examples”, “specific examples”, or “some examples” and other terms of reference are described to mean that the specific feature, structure, material, or characteristic described in conjunction with the embodiment or example is included in at least one embodiment or example of this invention. In this specification, schematic expressions of the above terms need not be directed to the same embodiments or examples. Moreover, the specific features, structures, materials, or characteristics described may be combined in any one or more embodiments or examples in a suitable manner. Furthermore, without contradicting each other, those skilled in the art may combine different embodiments or examples and features of different embodiments or examples described in this specification.

The infusion device may be used for delivering fluids such as medication and nutrient solutions. During the process of fluid delivery, the infusion tubing may become blocked or air bubbles may form. In order to ensure stable operation of the infusion device, it is necessary to monitor the condition of the fluid inside the infusion tubing.

Currently, the detection of blockages and bubbles in the infusion tubing is performed using a blockage sensor and an air bubble sensor, which detect these issues separately. The need for multiple sensors complicates the detection process and increases the production cost of the infusion pump.

In the method for monitoring the flow state of the infusion tubing of the invention, the data acquisition device is provided on the infusion tubing of the infusion device, and can acquire the state of fluid flow in the infusion tubing of the infusion device at different moments and form data during operation of the infusion device. By processing the signal strength data acquired by the data acquisition device at different moments, the infusion state of the infusion tubing can be determined, so that it can be determined whether the infusion tubing is in a normal state, a blocked state, or an air bubble state. The invention can realize the function of monitoring the flow state of the infusion tubing by acquiring data of the fluid flow state in the infusion tubing several times by the data acquisition device, which reduces the requirement for multiple data acquisition devices and reduces the types of signal strength data acquired by the data acquisition devices, thus achieving the purpose of simplifying the structure of the infusion system.

The method of monitoring the flow state of infusion tubing, the detection device of the infusion system, and the electronic device provided in the invention are described in detail below in connection with specific embodiments.

An embodiment of the invention proposes an infusion system, which may include an infusion device 200 and a data acquisition device 100. The infusion device 200 may be an infusion device such as a nutrient pump, which includes a controller, a driving mechanism, an extrusion component, and infusion tubing 210. The driving mechanism is connected to the extrusion component in a transmission mode, and the infusion tubing 210 is a tube for delivering a fluid (e.g., medicinal fluid or nutrient fluid), one end of which is in communication with a fluid storage device, and the other end of which is in communication with the patient. The extrusion component (e.g., a pump wheel) exerts a squeezing force on the infusion tubing 210. When the infusion device is working, the controller controls the driving mechanism to operate, thereby driving the extrusion component to squeeze the infusion tubing 210 and thereby driving the fluid to flow in the infusion tubing 210.

The data acquisition device 100 is provided in the infusion device 200 and is in contact with the infusion tubing 210 to determine the state of the fluid flow in the infusion tubing 210 of the infusion device and generate corresponding data. When the infusion device is in a state of continuous operation, the data acquisition device 100 may acquire the state of fluid flow within the infusion tubing 210 of the infusion device t different moments and generate multiple data signals.

In the detection device of the infusion system of the invention, the state detection function of the infusion tubing 210 can be realized by setting up only one data acquisition device 100 and acquiring the data of the fluid flow state within the infusion tubing 210 several times. The number of data acquisition devices 100 and the types of data required are reduced as compared with the prior art while also simplifying the structure of the infusion system.

In some embodiments, as shown in FIG. 2, the data acquisition device 100 of the invention may be an air bubble sensor. The infusion tubing 210 includes upstream tubing and downstream tubing, which are in communication with each other. The extrusion component (e.g., a pump wheel) is disposed between the upstream tubing and the downstream tubing, and the air bubble sensor is disposed at the upstream tubing. The air bubble sensor may include a transmitting portion 110 and a corresponding receiving portion 120. The transmitting portion 110 may transmit an ultrasonic signal into the infusion tubing 210, and the ultrasonic signal may reach the receiving portion 120 after passing through the infusion tubing 210 and be received by the receiving portion 120. Ultrasonic signals are poorly transmitted in air and are prone to attenuation, whereas they are better transmitted in fluid, with less attenuation. When the transmitting portion 110 transmits ultrasonic waves into the infusion tubing 210, if the infusion tubing 210 is filled with fluid, the ultrasonic signals will not have significant signal attenuation after passing through the infusion tubing 210, and the receiving section 120 can receive the ultrasonic signals with a stronger signal.

When there is an air bubble in the infusion tubing 210, however, and the ultrasonic signal will have a detectable attenuation when it passes through the air bubble in the infusion tubing 210. This will cause the signal strength received by the receiving section 120 to decrease. Similarly, when the infusion tubing 210 is constricted, which will cause at least a partial air gap that a portion of the transmitted ultrasound must cross (if the transmitter and receiver are mounted at a fixed distance between each other, due to the upper blockage of the tubing of the infusion tubing 210, it will cause the ultrasonic signals to attenuate relatively more, causing the signal value of the ultrasonic signals received by the receiving section 120 to decrease in strength.

Therefore, when the infusion tubing 210 is in a normal state, a blocked state, or an air bubble state, the signal strength data acquired by the data acquisition device 100 displays a difference, that is, the infusion tubing 210 being in a normal state, a blocked state, or an air bubble state has different data characteristics. Therefore, according to the signal strength data acquired by the data acquisition device 100, it can be determined that the infusion tubing 210 is in a normal state, a blocked state, or an air bubble state. It should be noted that when the data acquisition device 100 is located at the upstream tubing, the blocked state is the upstream blocked state, and when the data acquisition device 100 is located at the downstream tubing, the blocked state is the downstream blocked state.

In addition, the transmitting portion 110 and the receiving portion 120 may also be provided on the same side of the infusion tubing 210, and the ultrasonic signal emitted by the transmitting portion 110 may be reflected to the receiving portion 120 after reaching the infusion tubing 210.

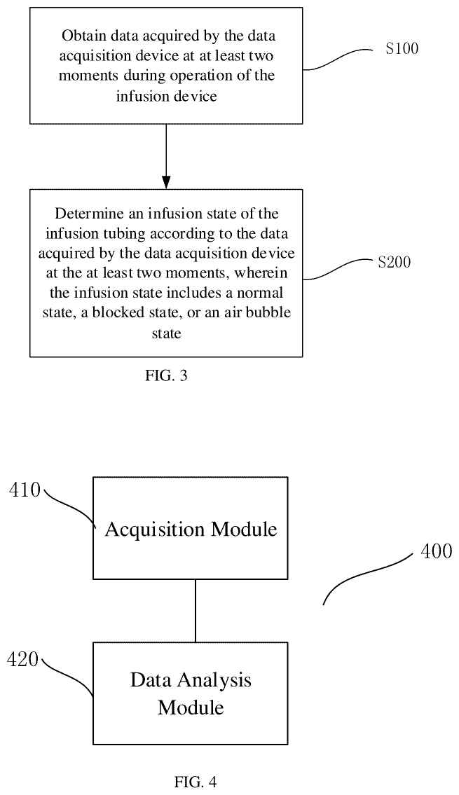

Embodiments of the invention also propose a method for monitoring the flow state of infusion tubing, which may be applied to the infusion system and the detection device of the infusion system described above. As shown in FIG. 3, the method includes the following steps.

S100, signal strength data acquired by the data acquisition device at at least two moments during operation of the infusion device is obtained. The operation period of the infusion device is when the infusion device is performing an infusion action, the infusion device has a fluid in the infusion tubing 210, and the infusion device drives the fluid in the infusion tubing 210 to flow along the infusion tubing 210. The data acquisition device 100 may acquire data at at least two moments, which data is related to the fluid flow condition within the infusion tubing 210. In an embodiment, the data may be an ultrasonic signal value.

S200, according to the signal strength data acquired by the data acquisition device at the at least two moments, an infusion state of the infusion tubing is determined. The infusion state may include a normal state, a blocked state, or an air bubble state.

The signal strength data acquired by the data acquisition device at different moments may reflect the flow condition of the fluid within the infusion tubing at different moments. By comparing and analyzing the signal strength data acquired by the data acquisition device at different moments, a change in the fluid within the infusion tubing may be obtained, so that it may be learned that the infusion state of the infusion tubing is in the normal state, the blocked state, or the air bubble state.

In some embodiments, as shown in FIG. 4, step S200 includes the following step:
When the data value acquired by the data acquisition device at a first moment in time is less than or equal to a first data threshold, determining the infusion state of the infusion tubing according to the change in the signal strength data acquired by the data acquisition device after the first moment.

The signal strength data acquired by the data acquisition device 100 is compared with the first data threshold. When the value indicated by the signal strength data acquired by the data acquisition device 100 is greater than the first data threshold, this indicates that there is almost no attenuation of the current ultrasonic signals compared to the ultrasonic signals in the normal condition. In this circumstance, the infusion state of the infusion tubing 210 is a normal state. When the data value acquired by the data acquisition device 100 is less than or equal to the first data threshold, however, this indicates that the current ultrasonic signals have a large amount of attenuation compared to the ultrasonic signals in the normal conditions, and the flow condition of the fluid in the infusion tubing 210 is abnormal, such that there may be a situation in which there is a blockage or an air bubble. Therefore, the infusion state of the infusion tubing 210 may be determined to be a blocked state or an air bubble state according to the change in the signal strength data acquired by the data acquisition device 100 after the first moment.

In some embodiments, the step of determining the infusion state of the infusion tubing according to the change in the signal strength data acquired by the data acquisition device after the first moment may further include the following step.

The infusion state of the infusion tubing is determined according to a rate of change of the signal strength data acquired by the data acquisition device after the first moment.

The rate of change of the data represents a change value of the data per unit of time. Regardless of whether the infusion tubing 210 is in the blocked state or the air bubble state, the signal strength data acquired by the data acquisition device 100 is subject to a certain degree of attenuation. However, the attenuation of the ultrasonic signal is different when the infusion tubing 210 is in the blocked state and the air bubble state. When the infusion tubing 210 is in the blocked state, the ultrasonic signal is slowly decreasing, and its attenuation rate is slow. When the infusion tubing 210 is in the air bubble state, the ultrasonic signal is rapidly decreasing, and its attenuation rate is fast. That is, the rate of change of the data when the infusion tubing 210 is in the blocked state is less than the rate of change of the data when the infusion tubing 210 is in the air bubble state.

A rate of change threshold associated with the rate of change of the ultrasonic signal is stored in the controller of the infusion device. When the rate of change of the signal strength data acquired by the data acquisition device 100 after the first moment is less than the rate of change threshold, it indicates that the ultrasonic signal is in a state of slow decline, and in this circumstance, the infusion state of the infusion tubing 210 is in the blocked state. When the rate of change of the signal strength data acquired by the data acquisition device 100 after the first moment is greater than the rate of change threshold, it indicates that the ultrasonic signal is in a state of rapid decline, and in this circumstance, the infusion state of the infusion tubing 210 is in the air bubble state. Thereby, the current infusion state of the infusion tubing can be determined to be the blocked state or the air bubble state according to the relationship between the rate of change of the signal strength data acquired by the data acquisition device 100 after the first moment and the rate of change threshold.

In some embodiments, the step of determining the infusion state of the infusion tubing according to the change in the signal strength data acquired by the data acquisition device after the first moment may further include the following steps:
A number and/or a duration of signal strength data measurements acquired by the data acquisition device after the first moment that lies between the first data threshold and the second data threshold is obtained.

The infusion status of the infusion tubing is determined according to the number and/or duration of the signal strength data measurements whose values fall between the first data threshold and the second data threshold.

During operation of the infusion device, the data acquisition device 100 may acquire data in real time, either continuously (at a chosen sampling rate) or according periodically, at a certain frequency, and the infusion state of the infusion tubing 210 may be determined by comparing the signal strength data acquired by the data acquisition device 100 with the first data threshold value and the second data threshold value. The first data threshold is greater than the second data threshold.

When the flow condition of the fluid within the infusion tubing 210 is normal, the signal strength data acquired by the data acquisition device 100 will be greater than the first data threshold. If the signal strength data acquired by the data acquisition device 100 up to a certain moment are all greater than the first data threshold, but the signal strength data acquired at a certain moment and after that moment are located between the first data threshold and the second data threshold, the moment is defined as the first moment, and the number of times that the data acquisition device 100 acquires data is recorded from the first moment.

After the first moment, when the signal strength data acquired by the data acquisition device 100 before another moment are all located between the first data threshold and the second data threshold, but the signal strength data acquired at the other moment and after the other moment are less than or equal to the second data threshold, the other moment is defined as the second moment. At the second moment, the data acquisition device 100 may stop recording the number of times the data acquisition device 100 acquires data. In this way, the number of times that the data acquisition device 100 acquires data in total during the time period from the first moment to the second moment is the number of data measurements acquired by the data acquisition device 100 that lies between the first data threshold and the second data threshold. According to the number of signal strength data measurements acquired by the data acquisition device 100 that lie between the first data threshold and the second data threshold, it may be determined whether the infusion tubing 210 is in the blocked state or in the air bubble state.

Regardless of whether the infusion tubing 210 is in the blocked state or the air bubble state, the signal strength sensed by the data acquisition device 100 is lower. When the infusion tubing 210 is blocked, resulting in the liquid not being smoothly input into the infusion tubing 210, then the amount of liquid in the infusion tubing 210 will gradually decrease, which causes the signal strength data acquired by the data acquisition device 100 to slowly decrease as well. When the infusion tubing 210 is in the blocked state, the data acquisition device 100 will then acquires a higher amount of data whose values fall between the first data threshold and the second data threshold, that is, first and second signal strength thresholds.

When an air bubble is present in the infusion tubing 210, the ultrasonic signal will be severely attenuated when passing through the air bubble. This causes the ultrasonic signal strength sensed by the data acquisition device 100 to rapidly decrease to the second data threshold. When the infusion tubing 210 is in the air bubble state, the data acquisition device 100 acquires a smaller amount of data whose values fall between the first data threshold and the second data threshold.

As can be seen from the above, the number of data measurements taken by the data acquisition device 100 that lie between the first data threshold and the second data threshold may be compared with a first predetermined value. When the number of data measurements taken by the data acquisition device 100 that fall between the first data threshold and the second data threshold is greater than the first predetermined value, this indicates that the infusion tubing 210 is in the blocked state. When the number of data measurements taken by the data acquisition device 100 between the first moment and the second moment is less than or equal to the first predetermined value, it indicates that the infusion tubing 210 is in the air bubble state. As a result, whether the infusion tubing 210 is in the blocked state or in the air bubble state may be determined according to the number of data measurements whose values fall signal strength data acquired between the first data threshold and the second data threshold—the greater the signal attenuation, the faster the drop will be, and the fewer measurements will be taken between the first and second thresholds.

In addition, a duration from the first moment to the second moment, i.e., a length of time of the signal strength data acquired by the data acquisition device that lies between the first data threshold (signal strength threshold) and the second data threshold (signal strength threshold), may also be calculated. Duration greater than a predetermined duration value indicates that the infusion tubing 210 is in the blocked state, and duration less than the predetermined duration value indicates that the infusion tubing 210 is in the air bubble state—as mentioned above, the measured signal strength will decrease faster for the air bubble state than for the blocked state, such that the time it takes for the signal strength to drop from the first threshold to the second threshold will be less. It will be appreciated that the duration from the first moment to the second moment may be directly counted, and the duration from the first moment to the second moment may also be determined by the relationship between the number of signal strength values signal strength data acquired by the data acquisition device 100 that lie between the first data threshold and the second data threshold and the frequency of data collection by the data acquisition device 100.

In some embodiments, the step of determining the infusion state of the infusion tubing according to the change in the data corresponding to received signal strength acquired by the data acquisition device after the first moment may further include the following step:
The infusion state of the infusion tubing is determined according to a difference between the signal strength data acquired by the data acquisition device after the first moment and the first data threshold. In an embodiment, it may include the following steps.

A difference between the signal strength data acquired by the data acquisition device after the first moment and the first data threshold is calculated.

Whether the difference is greater than or equal to the first difference threshold value is determined. If so, it is determined that the infusion tubing is in the air bubble state.

If not, whether the difference is greater than or equal to a second difference threshold is determined. If so, it is determined that the infusion tubing is in the blocked state. The second difference threshold is less than the first difference threshold.

The data acquisition device 100 may continuously acquire signal strength data after the first moment. When the difference between the signal strength data acquired by the data acquisition device after the first moment and the first data threshold is greater than or equal to the first difference threshold, it indicates that there exists, among the signal strength data acquired by the data acquisition device 100 after the first moment, signal strength data with a larger difference from the first data threshold, which in turn means that the signal strength data acquired by the data acquisition device 100 data has a large attenuation. When an air bubble appears in the infusion tubing 210, however, the signal strength data acquired by the data acquisition device 100 also decreases more significantly in a short period of time, and when the difference value is greater than or equal to the first difference threshold, this may indicate that an air bubble has appeared in the infusion tubing 210, and the infusion tubing 210 is in the air bubble state.

When the difference between the signal strength data acquired by the data acquisition device after the first moment and the first data threshold is greater than or equal to the second difference threshold and less than the first difference threshold, this indicates that the difference between the signal strength data acquired by the data acquisition device 100 after the first moment and the first data threshold is relatively small, and thus indicates that the magnitude of change of the signal strength data acquired by the data acquisition device 100 after the first moment is relatively small. When there is a blockage within the infusion tubing 210, the signal strength data acquired by the data acquisition device 100 is relatively more gently decreasing over a period of time, such that when the difference value is less than the first difference threshold value and greater than the second difference threshold value, it may indicate that there is a blockage within the infusion tubing 210, and the infusion tubing 210 is the blocked state.

It is to be noted that as long as, among the signal strength data acquired by the data acquisition device 100 after the first moment, there exists signal strength data whose difference with the first data threshold is greater than or equal to the first difference threshold, it can be determined that the infusion tubing 210 is in the air bubble state. For example, after the first moment, the difference between the signal strength data acquired by the data acquisition device 100 at the second moment and the first data threshold value may be greater than the first difference threshold value. If the signal strength data acquired by the data acquisition device 100 after the second moment goes up again, even if the difference between the signal strength data acquired by the data acquisition device 100 after the second moment and the first data threshold value is less than the first difference threshold value, the infusion tubing 210 is still considered to be in the air bubble state. In other words, as long as the signal acquired by the data acquisition device 100 after the first moment shows a relatively large attenuation, the infusion tubing 210 is considered to be in the air bubble state at the second moment regardless of how the subsequent signal strength data thereof changes, and corresponding alarm prompts are issued to avoid the situation of having air bubbles without any notification of this problem.

Similarly, after the first moment, the difference between the signal strength data acquired by the data acquisition device 100 at a third moment and the first data threshold may be located between the first difference threshold and the second difference threshold. If the signal strength data acquired by the data acquisition device 100 after the third moment rises again, even if the signal strength data acquired by the data acquisition device 100 after the third moment is greater than the first data threshold, the infusion tubing 210 is still considered to be in the blocked state and a corresponding alarm to avoid a situation of blockage omission.

Further, when the flow condition of the fluid in the infusion tubing 210 is normal, the signal strength data acquired by the data acquisition device 100 will be continuously and stably greater than the first data threshold. If the signal strength data acquired by the data acquisition device 100 are all greater than the first data threshold value during a time period, but suddenly the signal strength data acquired at the first moment is less than or equal to the first data threshold value, the infusion state of the infusion tubing 210 may be determined according to the change in the signal strength data acquired by the data acquisition device 100 after the first moment. If the signal strength data acquired by the data acquisition device 100 at a moment after the first moment is again consistently and steadily greater than the first data threshold, the infusion tubing 210 is considered to have returned to the normal state, and the infusion state of the infusion tubing 210 continues to be determined according to the signal strength data acquired by the data acquisition device 100 at at least two moments after that moment.

In some embodiments, the method for monitoring the flow state of the infusion tubing of the invention further includes the following step.

S300, an alarm message or a prompt message corresponding to the infusion state is output.

The infusion system may also include an alarm device 300 electrically connected to the data acquisition device 100. When the infusion tubing 210 is in the blocked state or in the air bubble state, this indicates that there is a problem with the flow of the liquid in the infusion tubing 210, which in turn indicates that there is a problem with the infusion device. In this circumstance, the alarm prompt is issued by the alarm device 300, and the user may be prompted to carry out corresponding procedures. The alarm device 300 may include a buzzer, an indicator light, and a display module. When the infusion tubing 210 is in the blocked state or in the air bubble state, the buzzer may emit an alarm sound corresponding to the blocked state and an alarm sound corresponding to the air bubble state. The display module may display textual information to indicate that the infusion tubing 210 of the infusion device is in the blocked state or the air bubble state. In addition, the display module may also display textual information to indicate that the infusion tubing 210 is in the normal state.

In some embodiments, different infusion states have different alarm information or prompt information, for example, the normal state, the blocked state, and the air bubble state correspond to indicator lights of different colors, and the current infusion state can be intuitively distinguished by displaying different colors in the indicator lights.

As shown in FIG. 4, the embodiment of the invention also proposes a device for monitoring the flow state of infusion tubing, which includes an acquisition module 410 and a data analysis module 420. The acquisition module 410 is configured to obtain a plurality of signal strength data acquired sensed by the data acquisition device 100 at at least two moments during operation of the infusion device. The acquisition module 410 is electrically connected to the data analysis module 420, so that the acquisition module 410 may transmit the plurality of signal strength data acquired to the data analysis module 420, which may analyze the plurality of data so as to determine whether the infusion tubing 210 is in a normal state, a blocked state, or an air bubble state.

According to an embodiment of the invention, the data acquisition device is an air bubble sensor.

According to an embodiment of the invention, the data analysis module 420 determines the infusion state of the infusion tubing according to the signal strength data acquired by the data acquisition device at at least two moments by: determining the infusion state of the infusion tubing according to the change in the signal strength data acquired by the data acquisition device after the first moment when the signal strength data acquired by the data acquisition device at a first moment is less than or equal to a first data threshold.

According to an embodiment of the invention, the data analysis module 420 determines the infusion state of the infusion tubing according to the change in the signal strength data acquired by the data acquisition device after the first moment by: determining the infusion state of the infusion tubing according to the rate of change in the signal strength data acquired by the data acquisition device after the first moment.

According to an embodiment of the invention, the data analysis module 420 determines the infusion state of the infusion tubing according to the change in the signal strength data acquired by the data acquisition device after the first moment according to the following step.

An acquisition unit acquires a number and/or a duration of signal strength data acquired by the data acquisition device after the first moment that lies between the first data threshold and the second data threshold.

A judgment unit determines an infusion state of the infusion tubing according to the quantity and/or duration of the signal strength data located between the first data threshold and the second data threshold.

According to an embodiment of the invention, the data analysis module 420 determines the infusion state of the infusion tubing according to the change in the signal strength data acquired by the data acquisition device after the first moment by: determining the infusion state of the infusion tubing according to a difference between the signal strength data acquired by the data acquisition device after the first moment and the first data threshold.

According to an embodiment of the invention, the detection device further includes an alarm module configured to issue an alarm message or a prompt message when it is determined that the infusion tubing is in the blocked state or in the air bubble state.

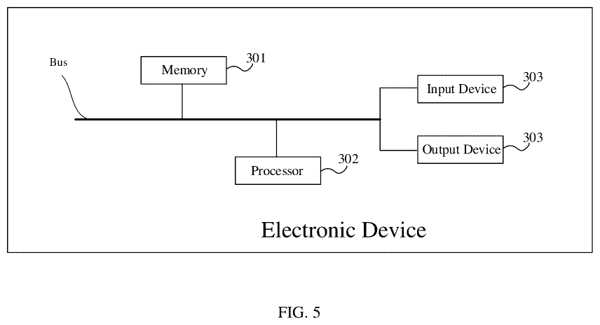

FIG. 5 is a schematic structural diagram of a nutrient infusion device provided by an embodiment of the invention. The components, their connections and relationships, and their functions shown herein are intended as examples only and do not limit the implementations of the invention described and/or claimed herein.

Embodiments of the invention provide a nutrient infusion device that includes: at least one processor 302 and a memory 301. The memory 301 is configured to stored computer-executable instructions, and the at least one processor 302 is configured to process the computer-executable instructions stored in the memory 301 to cause the nutrient infusion device to implement the above-described method of in-situ detection of consumables.

As shown in FIG. 5, the nutrient infusion device includes a processor 302 and a memory 301. The individual components are connected to each other utilizing different buses, and may be mounted on a common motherboard or otherwise mounted as desired. The processor 302 may process instructions executed on the nutrient infusion device, including instructions for graphical information stored in or on the memory 301 to be displayed on an external input/output device 304 (such as a display device coupled to the interface). In other embodiments, a plurality of processors 302 and/or a plurality of buses may be used with a plurality of memories 301 and a plurality of memories 301, if desired. A single processor 302 is used as an example in FIG. 10.

The memory 301 serves as a non-transitory computer-readable storage medium that can be used to store non-transitory software programs, non-transitory computer-executable programs, and modules, such as program instructions/modules corresponding to the method for monitoring the flow state of the infusion tubing in the embodiments of the invention (e.g., acquiring the state of the infusion tubing captured by the data acquisition device). The processor 302 executes various functional applications of the server as well as data processing by running the non-transient software programs, instructions, and modules stored in the memory 301, i.e., realizing the method for monitoring the flow state of the infusion tubing in the above-described method embodiment.

The nutrient infusion device may further includes: an input device 303 and an output device 304. The processor 302, the memory 301, the input device 303, and the output device 304 may be connected via a bus or otherwise, and the connection via a bus is used as an example in FIG. 10.

The input device 303 may receive input numeric or character information, as well as generate signal inputs related to user settings as well as function control of the nutrient infusion device. The input device 303 may be a touch screen, a keypad, a mouse, a plurality of mouse buttons, a trackball, or a joystick. The output device 304 may be a display device of the nutrient infusion device. The display device may include, but is not limited to, a liquid crystal display (LCD), a light emitting diode (LED) display, and a plasma display. In some embodiments, the display device may be a touch screen.

The nutrient infusion device of the embodiments of the invention may be used to perform the technical solutions in the above-described method embodiments of the invention, which are similar in terms of realization principles and technical effects, and will not be repeated herein.

Embodiments of the invention also provide a computer-readable storage medium having computer-executable instructions stored therein. The computer-executable instructions, when the processor processes the computer-executable instructions, causes the nutrient infusion device to implement any of the above-described methods.

In the several embodiments provided in this invention, it should be understood that the system, device, and method disclosed, may be realized in other ways. For example, the device embodiments described above are merely schematic, e.g., the division of the units described, is merely a logical functional division, and the actual implementation may be divided in other ways, e.g., a plurality of units or components may be combined or may be integrated into another system, or some features may be ignored, or not implemented. At another point, the coupling or direct coupling or communication connection between each other shown or discussed may be an indirect coupling or communication connection through some interface, device or unit, which may be electrical, mechanical or otherwise.

The units illustrated as separated components may or may not be physically separated, and components shown as units may or may not be physical units, i.e., they may be located in a single place or may also be distributed over a plurality of network units. Some or all of these units may be selected to fulfill the purpose of the embodiment scheme according to practical needs.

In addition, the various functional units in various embodiments of the invention may be integrated in a single processing unit, or each unit may be physically present separately, or two or more units may be integrated in a single unit. The above integrated units may be realized either in the form of hardware or in the form of software functional units.

Other embodiments of this disclosure will readily come to mind to those skilled in the art upon consideration of the specification and practice of the invention disclosed herein. This invention is intended to cover any variations, uses, or adaptations of this disclosure that follow the general principles of the present disclosure and include common knowledge or customary technical means in the art not disclosed herein. The specification and embodiments are to be regarded as exemplary only, and the true scope and spirit of this disclosure is indicated by the following claims.

It should be understood that this disclosure is not limited to the precise structure which has been described above and illustrated in the accompanying drawings, and that various modifications and alterations may be made without departing from its scope. The scope of this disclosure is limited only by the appended claims.

1. A method for monitoring the flow state of infusion tubing, applied to an infusion system, the infusion system comprising an infusion device and a data acquisition device communicatively connected to the infusion device, the data acquisition device being disposed on infusion tubing of the infusion device, wherein the method comprises:
transmitting ultrasound from a transmitting portion of the data acquisition device through the infusion tubing;
receiving the ultrasound in a receiving portion of the data acquisition device after the ultrasound has passed through the infusion tubing;
obtaining signal strength data of the received ultrasound acquired by the data acquisition device at at least two moments during operation of the infusion device; and
determining an infusion state of the infusion tubing according to the signal strength data acquired by the data acquisition device at the at least two moments, wherein the infusion state comprises a normal state, a blocked state or an air bubble state.

2. The method of claim 1, wherein the data acquisition device is an ultrasound transducer.

3. The method of claim 1, wherein the step of determining the infusion state of the infusion tubing according to the signal strength data acquired by the data acquisition device at the at least two moments comprises:
when the signal strength data acquired by the data acquisition device at a first moment is less than or equal to a first data threshold, determining the infusion state of the infusion tubing according to a change in the signal strength data acquired by the data acquisition device after the first moment.

4. The method of claim 3, wherein the step of determining the infusion state of the infusion tubing according to the change in the signal strength data acquired by the data acquisition device after the first moment comprises:
determining the infusion state of the infusion tubing according to a rate of change of the signal strength data acquired by the data acquisition device after the first moment.

5. The method of claim 3, wherein the step of determining the infusion state of the infusion tubing according to the change in the signal strength data acquired by the data acquisition device after the first moment comprises:
obtaining at least one of: a number and a duration of signal strength measurements acquired by the data acquisition device after the first moment that located between a first data threshold and a second data threshold; and
determining the infusion state of the infusion tubing according to at least one of the number and the duration of signal strength measurements located between the first data threshold and the second data threshold.

6. The method of claim 3, wherein the step of determining the infusion state of the infusion tubing according to the change in the signal strength data acquired by the data acquisition device after the first moment comprises:
determining the infusion state of the infusion tubing according to a difference between the signal strength data acquired by the data acquisition device after the first moment and the first data threshold.

7. The method of claim 1, further comprising:
outputting an alarm message or a prompt message corresponding to the infusion state.

8. A nutrient infusion device, comprising: at least one processor and a memory;
the memory is configured to store computer-executable instructions; and
the at least one processor is configured to process the computer-executable instructions stored in the memory to cause the nutrient infusion device to:
obtain signal strength data acquired by the data acquisition device at at least two moments during operation of the infusion device, said signal strength data corresponding to the strength of ultrasound transmitted through infusion tubing of the infusion device;
determine an infusion state of the infusion tubing according to the signal strength data acquired at the at least two moments, wherein the infusion state comprises a normal state, a blocked state or an air bubble state.

9. The nutrient infusion device of claim 8, wherein the data acquisition device is an ultrasound transducer.

10. The nutrient infusion device of claim 8, wherein the nutrient infusion device determines the infusion state of the infusion tubing according to the signal strength data acquired by the data acquisition device at the at least two moments by:
determining the infusion state of the infusion tubing according to a change in the signal strength data acquired by the data acquisition device after the first moment when the signal strength data acquired at a first moment is less than or equal to a first data threshold.

11. The nutrient infusion device of claim 10, wherein the nutrient infusion device determines the infusion state of the infusion tubing according to the change in the signal strength data acquired by the data acquisition device after the first moment by:
determining the infusion state of the infusion tubing according to a rate of change of the signal strength data acquired by the data acquisition device after the first moment.

12. The nutrient infusion device of claim 10, wherein the nutrient infusion device determines the infusion state of the infusion tubing according to the change in the signal strength data acquired by the data acquisition device after the first moment by:
obtaining at least one of a number and a duration of signal strength measurements acquired by the data acquisition device after the first moment that located between a first data threshold and a second data threshold;
determining the infusion state of the infusion tubing according to at least one of the number and the duration of the signal strength measurement that fall between the first data threshold and the second data threshold.

13. The nutrient infusion device of claim 10, wherein the nutrient infusion device determines the infusion state of the infusion tubing according to the change in the signal strength data acquired by the data acquisition device after the first moment by:
determining the infusion state of the infusion tubing according to a difference between the signal strength data acquired by the data acquisition device after the first moment and the first data threshold.

14. The nutrient infusion device of claim 10, wherein the nutrient infusion device is further configured to:
output an alarm message or a prompt message corresponding to the infusion state.

15. A computer-readable storage medium having computer instructions stored therein, wherein the computer instructions when processed by the processor are configured to process the computer-executable instructions to cause a nutrient infusion device to:
obtain signal strength data acquired by the data acquisition device at at least two moments during operation of the infusion device;
determine an infusion state of the infusion tubing according to the signal strength data acquired by the data acquisition device at the at least two moments, wherein the infusion state comprises a normal state, a blocked state or an air bubble state.
